(19) United States
(12) Patent Application Publication
(10) Pub. No.: US 2023/0110189 A1
(43) Pub. Date: Apr. 13, 2023

(54) FLUID FLOW CONTROL OF A BLOOD TREATMENT DEVICE

(71) Applicant: FRESENIUS MEDICAL CARE DEUTSCHLAND GMBH, Bad Homburg (DE)

(72) Inventor: Tobias IRRGANG, Aubstadt (DE)

(73) Assignee: FRESENIUS MEDICAL CARE DEUTSCHLAND GMBH, Bad Homburg (DE)

(21) Appl. No.: 17/798,567

(22) PCT Filed: Jan. 25, 2021
(86) PCT No.: PCT/EP2021/051610

§ 371 Date: Aug. 10, 2022

(30) Foreign Application Priority Data
Feb. 17, 2020 (DE) 10 2020 104 101.2

(51) Int. Cl. A61M 1/16

(52) U.S. Cl. CPC A61M 1/1656; A61M 1/1603; A61M 2205/3334; A61M 2205/3327; A61M 2205/3317; A61M 2205/3375; A61M 2205/3344

(57) ABSTRACT

The invention relates to a blood treatment device, having the following:

a fluid line system (10) for guiding a fluid flow comprising a line portion (14), wherein the line portion (14) can be formed as a closed fluid system, and comprising at least one first concentrate supply line (26) for supplying a first concentrate solution; a fluid pump (11) for conveying the fluid in the fluid line system (10); a determining means (13; 23; 33; 29) for capturing a state of the fluid line system (10); a control unit for controlling the fluid flow; characterized in that the control unit is configured in such a way that the line portion (14) only forms a closed fluid system when the state captured by the determining means (13; 23; 33; 29) meets a predetermined condition.

### TECHNICAL FIELD

The present invention relates to a blood treatment device as well as a method for controlling a fluid flow in a blood treatment device.

### BACKGROUND

The term blood treatment device is to be understood, inter alia, as a dialysis machine. Dialysis machines are frequently used in dialysis centers to treat chronic kidney disease. In addition to high safety requirements, timing plays a special role when using dialysis machines in dialysis centers.

In the case of this use of blood treatment devices, in particular in the case of dialysis treatments, in particular the adherence to exact schedules is essential for the below-mentioned reasons. On the one hand, the dialysis machines, which are used in the case of dialysis treatments in a dialysis center, are routinely assigned to the individual treatments of various dialysis patients in such a way that three persons can be treated per day at one dialysis machine with a treatment duration of 4 to 5 hours.

The length of time required between the treatments for preparing the person to be treated, the patient, as well as other steps, such as, e.g., the length of time required for disinfecting the dialysis machine, is subject to a constant time optimization. If a treatment is delayed, the subsequent treatments and thus also the patients' schedules but also the schedules of the health-care staff, the nursing staff or nephrologist, have to be changed. Consequences of such a change may lead to overtime and stress to the health-care staff as well as to costs for the respective treatment center, for example due to an unfavorable utilization of the dialysis machines.

Due to this tight timing, not only the time between the treatments should be kept as short as possible, but interruptions during a treatment need to also be kept as short as possible.

If the subsequent treatments are delayed due to frequent interruptions of a treatment, this does not only lead to disadvantages for the treatment center and staff, delays of a treatment also have disadvantageous impacts on the patient.

If treatments are delayed, for instance to the next day or even by several days due to holidays and weekends, this may lead to consequences, which are dangerous to health. Excess water levels as well as the increasing poisoning due to the build-up of urinary substances have a critical impact on the patient's health.

In addition, long treatments have a negative impact on the patient's well-being. It is the goal to make it possible for a patient to have a daily life, which his as normal as possible, in spite of regular treatments. The treatment is to thus not take up more time than necessary.

In addition to the above-listed time-critical treatment situation, it is advantageous to constantly monitor the proper operation of the blood treatment device, in order to be able to ensure the patient's safety.

Various safety systems are known to ensure the safety during a dialysis treatment. To check the proper operation, the treatment can be interrupted when carrying out some safety tests. For example a pressure holding test is known as safety system from DE 4239937 C2.

At the beginning of this pressure holding test, a predetermined amount of fluid is conveyed into the dialysis fluid circuit. An outflow valve is closed during the pressure holding test, so that a predetermined pressure can be built up. During this test, the dialysis fluid circuit is in an advanced bypass, so that the fluid cannot reach into the dialyzer.

The curve of the fluid pressure in the dialysis fluid circuit can be determined from the pressure value measured at one or several points in the dialysis fluid circuit. The pressure holding test is performed, for example, for a length of time of 8 seconds and periodically during the treatment.

If, for performing the pressure holding test, a non-physiological (unphysiological) fluid now reaches into the dialysis fluid, this fluid has to be removed from the dialysis fluid circuit before continuing the treatment, in order to prevent that unphysiological fluid reaches the patient.

To detect an unphysiological fluid, a conductivity sensor for monitoring the composition of the fluid is provided for example in the dialysis fluid circuit. If unphysiological fluid is captured by means of the conductivity sensor, this fluid has thus already reached the dialysis fluid circuit. For this purpose, the conductivity sensor can compare the measured value to a setpoint value or setpoint value range, and the blood treatment device or the control thereof, respectively, can detect an unphysiological fluid in the case of a deviation.

To remove the unphysiological fluid from the dialysis fluid circuit, a time-sensitive flushing should be performed after a positive detection of an unphysiological fluid, wherein the interruption of the treatment is extended. The above-specified negative impacts can result therefrom

Efforts are made in the prior art to keep the interruption of the treatment due to the pressure holding test low. For example, EP 1327457 B1 discloses a method for the detection of leakages in the fluid system, which allows for the monitoring without interruption of the blood treatment due to the performance of a pressure holding test. A pressure holding test is thus only performed when there is a high likelihood of a leakage. If a high likelihood for a leakage is detected, the treatment is thus also interrupted in response to this method, and a pressure holding test is performed.

The present application is thus based on the object of keeping the interruption of the blood treatment, in particular due to the performance of a pressure holding test, as short as possible and to simultaneously ensure the safety of the patient.

### SUMMARY OF THE INVENTION

The object on which the invention is based is solved by means of the blood treatment device according to claim 1, and by means of the method for controlling a fluid flow in a blood treatment device according to claim 13. Advantageous further developments and embodiments are subject matter of the dependent claims.

According to the invention, a blood treatment device is provided, which has a fluid line system for guiding a fluid flow comprising a line portion and at least one first concentrate supply line for supplying a first concentrate solution. The line portion can thereby be formed as a closed fluid system. The blood treatment device further has a fluid pump for conveying a fluid in the fluid line system, as well as a determining means for capturing a state of the fluid line system, and a control unit for controlling the fluid flow. The blood treatment device is characterized in that the control unit is configured in such a way that the line portion only forms a closed fluid system when the state captured by the determining means meets a predetermined condition.

According to the invention, a closed fluid system is a fluid system, in which neither an inflow nor an outflow of a fluid takes place. After a certain time in the closed fluid system, the fluid thereby comes to a standstill, that is, a fluid flow does no longer take place, so that the dynamic pressure prevailing in the closed fluid system approaches zero and the total pressure corresponds approximately to the static pressure.

The fluid line system thereby has a line portion, which can be formed as a closed fluid system. In other words, a partial portion of the fluid line system can be separated in such a way that the fluid can be held in this partial portion.

In addition to the line portion, the fluid line system has a concentrate supply line. This concentrate supply line is directly or indirectly connected to the line portion in such a way that a fluid conveyed via the concentrate supply line, for instance a concentrate solution, can reach into the line portion.

The control unit controls the fluid flow by systematically controlling at least one fluid pump and/or locking elements arranged in the blood treatment device. These elements, which can be controlled by the control unit, can thereby also be arranged outside of the blood treatment device.

The determining means captures a state of the fluid line system. The determining means in particular captures a state of the fluid, which is present in the fluid line system. The determining means can thereby be a fluid sensor. In the alternative or simultaneously, the determining means can be a sensor for capturing a position of a connecting means, that is, the determining means can have several physical sensors, for example a conductivity sensor and/or a conductance sensor and/or a magnetic sensor and/or a contact sensor. In other words, the determining means can have several elements. The determining means can thereby also have only two fluid sensors, which are arranged at different positions in the fluid line system.

The line portion is closed, that is, a closed fluid system is formed, only when the state of the fluid line system, which is captured by the determining means, meets a predetermined condition.

The predetermined condition of the state of the fluid line system can thereby specify a value range, which is defined by an upper and/or lower limit. However, the predetermined condition can detect even only a presence. The predetermined condition can be defined, for example, as a presence of a fluid or as a presence of a connecting means.

If the state does not meet the predetermined condition, this means that the line portion does not form a closed fluid system. In other words, fluid is guided out of the line portion. The fluid can thereby either be guided out of the line portion, that is, an outflow valve is open, while fluid can simultaneously be supplied to the line portion, that is, an inlet valve is open as well. In the alternative, the fluid can only be guided out of the line portion while no fluid is supplied to the line portion, in other words, the line portion is emptied of the fluid. A closed fluid system is only formed when the state captured by the determining means, that is, the presence of a fluid and/or the composition of a fluid and/or the position of a connecting means, meets a predetermined condition.

Due to the blood treatment device according to the claims, the interruption of the treatment is kept as short as possible. This is achieved, inter alia, in that a closed fluid system is formed only when the fluid value meets the predetermined condition. It is thus prevented that a pressure holding test is performed, even though the conditions necessary for this are not met. The performance of a pressure holding test at an early point in time can thus be prevented, for example when an unphysiological fluid is captured.

As a result, a performance of the pressure holding this is avoided when a predetermined condition is not met, for example when an unphysiological fluid is present. In addition to time savings, the patient safety is also ensured thereby. Unphysiological fluid is not held in the blood treatment device. The likelihood that unphysiological fluid reaches the patient is further reduced.

According to a further development of the blood treatment device, the blood treatment device can further have a first concentrate sensor for capturing a first concentrate value prevailing in the first concentrate supply line, wherein the determining means comprises the first concentrate sensor. In the case of this further development, the predetermined, first condition is only met when the first concentrate value meets a first concentrate value condition. In addition or in the alternative, the blood treatment device can have a mixed fluid sensor for capturing a mixed fluid value prevailing in the fluid line system downstream from the first concentrate supply line, wherein the mixed fluid sensor can be the determining means. In the case of this additional or alternative further development, the predetermined condition is only met when the mixed fluid value meets a mixed fluid value condition.

In other words, the determining means can be a concentrate sensor and/or a mixed fluid sensor. This means that, in a first alternative, the predetermined condition is only met when the first concentrate value as well as the mixed fluid value each meet a predetermined condition. The predetermined condition for the mixed fluid value and the concentrate value can thereby each be a different condition.

The determining means as concentrate sensor and/or mixed fluid sensor for capturing a state of the fluid line system, here a property of the fluid, can thereby be a sensor, which captures one or several chemical compositions of the fluid. However, the determining means can thereby also be an indicator, which only displays a presence of the fluid, without capturing a composition of the fluid thereby.

If the determining means captures a property of the fluid and if this captured fluid property does not lie within a predetermined value range, which predetermines the predetermined condition in this case, the predetermined condition is thus not met. The composition of the fluid is to be understood as fluid property in terms of this invention. The composition can thereby cover a range, which starts at zero, whereby zero means that no fluid is present.

In a second alternative, the state only meets the predetermined condition when the concentrate value as well as the mixed fluid value meet a respective predetermined condition. In other words, the situation may occur that the state in the second alternative does not meet the predetermined condition, even though the concentrate value or the mixed fluid value meets the respective predetermined condition. If the concentrate value and/or the mixed fluid value does not meet a respective predetermined condition, this means in terms of the invention that the state of the fluid line system, captured by the concentrate sensor of mixed fluid sensor, does not meet the predetermined condition.

The first concentrate value in terms of this invention refers to a property of the composition of a concentrate solution, which is present in the first concentrate supply line. The concentrate solution is thereby a fluid, which is brought together with a further fluid, which is supplied to the blood treatment device, and thus forms a mixed fluid. The mixed fluid value in terms of this invention thus refers to a property of the composition of the mixed fluid. According to the invention, the mixed fluid is thus only formed downstream from the concentrate supply line.

With the separate capture of properties of the fluid at different points in the fluid line system, the likelihood that an unphysiological fluid is captured quickly is increased. The likelihood that an unphysiological fluid is detected is simultaneously increased by the capture of the fluid property at different points.

According to a further development, the blood treatment device can further have a connecting means for transferring the first concentrate solution into the first concentrate supply line as well as a chamber, into which the connecting means can be introduced. The blood treatment device can further have a connecting means sensor for capturing a position of the connecting means. In the case of this further development, the determining means thereby comprises the connecting means sensor, and the predetermined condition is only met when the position of the connecting means meets a position condition.

A connecting means in terms of the invention is a means, with which a transfer of a fluid is made possible. The connecting means is thereby a tubular element, which can also be moved relative to the chamber. In other words, the connecting means can be a flexible connection, for instance a hose arrangement, by means of which a fluid can be conveyed, or a rigid connecting element, for instance a suction wand. The fluid can thereby be pushed into the concentrate supply line by applying negative pressure, can be conveyed through a pump or drawn in by generating a negative pressure.

A connecting means sensor according to the invention can capture the position of the connecting means. The connecting means sensor can thereby capture the position of the connecting means in a positive manner as a presence of the connecting means. A capture of the position in terms of the invention, however, can also mean a negative capture, so that the connecting means sensor captures that the connecting means is not present. The connecting means sensor can be a contact sensor, light sensor or magnetic sensor.

A chamber in terms of the invention is a cavity, which is formed in the blood treatment device and which has an opening to the surrounding area. The connecting means can be introduced into the chamber via this opening. With regard to its dimensions, the chamber is thus adapted to the connecting means in such a way that the connecting means is prevented from falling out. A locking mechanism, for instance, which interacts with the connecting means, can in particular be formed on the chamber.

If it is captured with the help of the connecting means sensor that the connecting means is not at a position, at which physiological fluid can be conveyed, the state, in which an unphysiological fluid is present in the blood treatment device, can thus be detected even before a capture of the unphysiological fluid by means of a fluid sensor, the mixed fluid sensor or concentrate sensor.

With this further development, the likelihood that an unphysiological fluid is detected early is further increased. The performance of a pressure holding test in the case of unphysiological conditions, for example the presence of an unphysiological fluid, can be prevented. This results in particular in the advantage of time savings, because the pressure holding test is interrupted as early as possible or a time-intensive flushing process can be prevented in response to a corresponding control.

According to a further development, the blood treatment device can further have a means for capturing a concentrate supply mode. In the case of this further development, the predetermined condition is only met when the position of the connecting means meets the position condition and when the captured concentrate supply mode is a concentrate supply mode, in which the concentrate supply takes place via the connecting means in the detected position.

A concentrate supply mode in terms of the invention specifies the mode of the concentrate supply of the blood treatment device. A concentrate supply can thereby take place via a concentration solution provided in liquid form or via a dry concentrate. In the case of the first concentrate supply mode, the amount of concentrate, which is necessary for a treatment, is delivered to the blood treatment device in liquid form in a concentrate container. The concentrate is thus already prepared in a solution.

In other words, the first concentrate supply mode can be a mode, in the case of which the supply of the first concentrate solution into the first concentrate supply line takes place via the connecting means, wherein the connecting means in the first concentrate supply mode for supplying the first concentrate solution is not introduced in the chamber.

In the case of another concentrate supply mode, the concentrate, which is provided in liquid form, is supplied via a central supply. For this purpose, a central supply connection is provided on the blood treatment device. A central supply line is connected via the central supply connection. In the case of this concentrate supply mode, the concentrate reservoir is provided at a distance from the blood treatment device, for instance in a different room.

In the case of a further alternative of the concentrate supply, the concentrate can be assigned to the blood treatment device as dry concentrate in a concentrate bag. For example, the dry concentrate can be attached to the blood treatment device in a concentrate bag.

If the concentrate supply mode is captured in addition to the position of the connecting means in the case of the blood treatment device, the likelihood that an unphysiological fluid is in fact present, is increased thereby. The likelihood that the event occurs that the determining means captures that the fluid line system does not meet the predetermined condition even though no unphysiological fluid is present, is thus reduced. In other words, an unnecessary prevention of the pressure holding test is avoided.

According to a further development, the blood treatment device can further have a second concentrate supply line for supplying a second concentrate solution as well as a second concentrate sensor for capturing a second concentrate value in the second concentrate supply line. In the case of this further development, the determining means comprises the second concentrate sensor, and the predetermined condition is only met when the second concentrate value meets a second concentrate value condition.

The second concentrate supply line in terms of the invention is a further supply line, by means of which a fluid, more precisely a concentrate solution, is supplied. The second concentrate supply line can thereby merge with the first concentrate supply line first, so as to subsequently be supplied to a further fluid, for instance a permeate, and so as to form the mixed fluid. In the alternative, the first or the second concentrate supply line can be merged with the further fluid first, so as to subsequently be merged with the first or second concentrate solution. In the alternative, the first and second concentrate solution can be supplied simultaneously, that is, via a mixing point, and can thus form the mixed fluid.

In the case of this further development, the predetermined condition is met when the second concentrate value meets a second concentrate value condition. The second concentrate value condition is a condition for a composition of the second concentrate. The second concentrate value condition can thereby differ from the first concentrate value condition.

According to a further development, the connecting means sensor can have a magnetic sensor or a contact sensor, in particular a Hall sensor or a mechanical switch.

According to a further development, a connecting means sensor can be arranged on the blood treatment device on the chamber of the blood treatment device. In addition or in the alternative, the connecting means sensor can be formed in such a way that it can be attached to a concentrate container, which can be delivered to the blood treatment device.

In terms of the invention, the connecting means sensor can be arranged on the chamber of the blood treatment device. In other words, the connecting means sensor arranged on the chamber of the blood treatment device captures whether the connecting means is introduced in the chamber or whether the connecting means is at least partially located in the chamber of the blood treatment device, respectively. The connecting means sensor can thereby be a contact sensor, for instance a Hall sensor.

In the alternative or in addition, the connecting means sensor can be located at a location at a distance from the chamber. The connecting means sensor can thus capture that the connecting means is located at a distance from the chamber, and thus not in the chamber.

Sensor arrangements, which are arranged on the connecting means as well as on the chamber, are thus also captured by the solution according to the invention. A magnet can thus be attached to the connecting means or at a location at a distance from the blood treatment device, for instance a concentrate container, which is delivered to the blood treatment device. The corresponding magnetic sensor can be arranged accordingly on the concentrate container or the connecting means as counter piece. A Hall sensor, which is arranged on the chamber of the blood treatment device or at a location at a distance from the blood treatment device, for instance a concentrate container, is likewise captured as connecting means sensor.

According to a further development, the first and/or the second concentrate sensor can be a conductance sensor or a conductivity sensor or an ultrasonic sensor. In addition or in the alternative, the mixed fluid sensor can be a conductance sensor or a conductivity sensor or an ultrasonic sensor. Types of concentrate sensors, which differ from those used for the mixed fluid sensor, can be used for the concentrate sensors. It is thus possible to increase the system safety due to different types of sensors and thus control or control mechanisms, respectively.

According to a further development, the blood treatment device can further have a line branch. The line branch is thereby formed parallel to at least one partial portion of the line portion, so that a fluid flow can be connected via the line branch in the case of a fluid flow via the line portion. A dialyzer is furthermore arranged in the line branch.

The line branch in terms of the invention is a fluid line portion, which runs parallel to a partial portion of the fluid line system. The line branch is thereby formed parallel to a partial portion of the line portion, so that no fluid flow takes place via the line portion in the case of a fluid flow via the line portion.

According to this further development of the blood treatment device, the blood treatment device has a dialyzer in the line branch. The blood treatment device is formed as dialysis machine in this case.

By forming the dialyzer in the line branch, the safety can be increased. The dialyzer can in particular be uncoupled from the fluid flow in that the fluid is guided via the part of the line portion, which runs parallel to the line branch. The line portion can furthermore be formed as a closed system. By monitoring the pressure curve in this system, the operation of the system components, for example of valves, can be checked in a closed system, in which no inflow and outflow of fluid takes place.

According to a further development, the blood treatment device further has locking elements in the fluid line system. The control unit can thereby be configured in such a way that the line portion is formed as a closed fluid system by controlling at least the locking elements.

According to a further development of the invention, the blood treatment device further has a pressure sensor for capturing a pressure value in the line portion. In the case of this further development, the control unit is thereby configured to only perform a pressure holding test in the line portion in the line portion when the state captured by the determining means meets the predetermined condition. In the case of this pressure holding test, a pressure of, preferably from 600 to 800 mmHg, more preferably from 700 to 750 mmHg, is applied to the fluid in the line portion, and the pressure values in the line portion are captured during a predetermine time period, and a conclusion can be drawn from a change of the pressure values to a leakage in the line portion.

The line portion can be formed as a closed system. A pressure holding test can be performed in the closed system. A pressure holding test in terms of the invention is a method, in the case of which the dialyzer is in each case separated from the dialysis fluid circuit for a short time interval at regular time intervals, for example 8 seconds, during a treatment. The pressure curve during this short time interval is captured by detection of signals by means of at least one pressure sensor. A conclusion to the state of the system and system components can subsequently be drawn from the captured pressure values, in particular from the curve of the pressure values, and a possible leakage can be identified. The pressures prevailing in the dialysis fluid circuit during the pressure holding test, particularly preferably +725 mmHg, thereby lie above the pressures, which are present during the treatment.

According to a further development, the blood treatment device can have at least one first concentrate pump for conveying the first concentrate solution.

The concentrate pump for conveying the first concentrate solution can be arranged in the first concentrate supply line. In the alternative, the concentrate pump can also be arranged at a distance from the first concentrate supply line, and can convey the first concentrate solution, for example by generating negative pressure in the first concentrate supply line.

A method for controlling a fluid flow in a blood treatment device comprising the above-mentioned advantages is further proposed.

The method for controlling a fluid flow in a blood treatment device, wherein the blood treatment device has a fluid line system for guiding a fluid flow comprising a line portion, which can be formed as a closed fluid system, and at least a first concentrate supply line for supplying a first concentrate solution, has the following steps: detecting a state of the fluid line system by means of a determining means; and matching the state to a predetermined condition.

The method is thereby characterized by allowing that the line portion only forms a closed fluid system when the state of the fluid line system meets the predetermined condition.

A further development of the method can be provided thereby, wherein the blood treatment device has a first concentrate sensor as determining means for capturing a first concentrate value prevailing in the first concentrate supply line, and wherein the predetermined condition is only met when the concentrate value meets a concentrate value condition and/or the blood treatment device has a mixed fluid sensor as determining means for capturing a mixed fluid value in the fluid line system downstream from the concentrate supply line, and wherein the predetermined condition is only met when the mixed fluid value meets a mixed fluid value condition.

A further development can thereby be provided in addition to or as an alternative for the method, wherein the blood treatment device has a chamber and a connecting means, which can be introduced into the chamber, as well as a connecting means sensor as determining means for capturing a position of the connecting means, further having detecting a position of the connecting means by means of the connecting means sensor, wherein the predetermined condition is only met when the position of the connecting means meets a position condition.

Due to this solution according to the claims, an unphysiological fluid is in particular detected as early as possible, for instance before it has reached the dialysis fluid circuit. The interruption of the treatment can thus be kept as short as possible, in that the pressure holding test is ended immediately or the beginning of a pressure holding test is avoided, respectively, as soon as an unphysiological fluid is detected. As a result, a performance of a pressure holding test can be avoided when unphysiological conditions are at hand, that is, when the conditions for the fluid value are not met. In addition to the time advantage, the patient safety is additionally ensured, because no closed system is formed, in which pressure is applied to unphysiological fluid.

The control can furthermore be designed in such a way that a time-consuming flushing process of the dialysis fluid circuit is prevented, because the unphysiological fluid is detected at an earliest possible point in time, for example prior to reaching the dialysis fluid circuit.

The above-described features and functions of the present invention as well as further aspects and features will be described in more detail below on the basis of a detailed description of preferred embodiments with reference to the enclosed figures. Identical features/elements and features/elements comprising the same function are identified with the same reference numerals in the figures.

### BRIEF DESCRIPTION OF THE DRAWINGS

In which

FIG. 1 shows a diagram of a blood treatment device;

FIG. 2 shows a section of a concentrate supply arrangement of the blood treatment device;

FIG. 3 shows a flow chart of a method for controlling a fluid flow in a blood treatment device.

### DETAILED DESCRIPTION OF AN EXEMPLARY EMBODIMENT

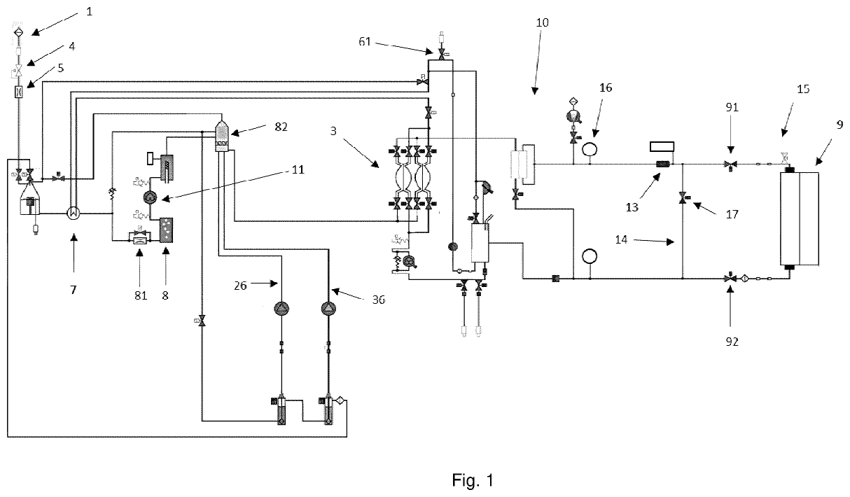

With reference to FIG. 1, a first embodiment will be described below. FIG. 1 thereby shows a simplified diagram of a blood treatment device. In the exemplary embodiment shown in FIG. 1, the blood treatment device is a dialysis machine.

In the case of the blood treatment device shown in FIG. 1, the blood to be treated flows through a blood chamber of a dialyzer 9 in an extracorporeal blood circuit. A dialysis fluid flows through a dialysis fluid circuit and, in the counter flow principle, through a dialysis fluid chamber of the dialyzer 9. The blood chamber as well as the dialysis fluid chamber are thereby separated by means of a semi-permeable membrane.

Substances to be removed from the blood pass through the semi-permeable membrane into the dialysis fluid and are thus discharged through the dialysis fluid, now referred to as dialysate. An excess amount of fluid can simultaneously be ultrafiltered out of the blood via a pressure gradient. The amount of fluid to be removed is conveyed with the help of an ultrafiltration pump.

To purify a patient's blood, blood is drawn from the patient via an arteriovenous fistula by means of a shunt and is guided into the extracorporeal blood circuit. The conveying of the blood thereby takes place with the help of a blood pump, which is not illustrated here. The purified blood leaves the dialyzer 9 and is subsequently supplied to the patient again.

The fluid supply of the blood treatment device takes place via a dialysis water connection 1, a pressure reducing valve 4 connected downstream, which reduces the pressure for example to approximately 0.5 bar, as well as an intake regulator 5. Permeate, that is, softened and filtered water, is supplied via the dialysis water connection 1. In the exemplary embodiment described here of the blood treatment device as a dialysis machine, permeate is the base fluid.

After passing through the dialyzer 9, the dialysate is supplied to an outflow via a dialysis fluid discharge line and an outflow valve 61. The heating of the fresh dialysis fluid can thereby take place via a heat exchanger 7 through the dialysate, so as to subsequently be further heated, for instance by means of a heating coil or a heating rod. In addition, the permeate is subjected to a degassing in a degassing chamber 8. To convey the solution of air, the permeate is subjected to a negative pressure by means of a degassing regulator 81 for this purpose. Due to the temperature increase and pressure reduction, air can thus escape in bubble form via an air separator 82 connected downstream.

The permeate is conveyed with the help of a fluid pump 11. The fluid pump 11 can thereby be formed, for example, as gear pump, membrane pump or the like. If the fluid pump 11 is formed as gear pump, a bypass is formed around the gear pump, so that the gear pump does not need to be stopped when preventing the fluid flow. As described above, the dialysate is supplied to an outflow via a flow pump and a balance chamber 3 following in the flow direction.

After the degassing of the base fluid, here the permeate, the mixed fluid, here the dialysis fluid, is created by admixing at least one concentrate solution. To provide the fresh dialysis fluid, permeate supplied via the dialysis water connection 1, and for example two concentrate solutions, for instance a bicarbonate concentrate solution and an acid concentrate solution, supplied for example from concentrate containers, which are not illustrated here, is thus mixed.

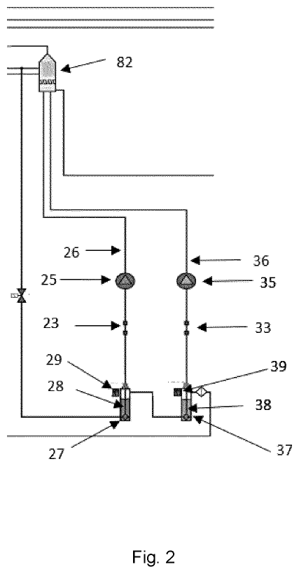

As illustrated in FIG. 2, the concentrate solutions can be conveyed via concentrate pumps 25, 35. The concentrate pumps 25, 35 can be formed, for example, as reciprocating pumps, membrane pumps or gear pumps. The proportioning, that is, the mixture of acid concentrate and bicarbonate with permeate at a predetermined ratio, can take place volumetrically or controlled by conductivity. In the case of the volumetric proportioning shown in this exemplary embodiment, the supplied volume is reached via a clocked supply by means of the concentrate pumps 25, 35, for example reciprocating pumps.

In the alternative, however, the proportioning can also take place in a conductivity-controlled manner, wherein the proportioning is controlled by means of conductivity sensors here. The supply of concentrate is increased thereby, until the desired conductivity is reached. The dialysis fluid created by means of the mixing subsequently flows through a part of the balance chamber 3 and thus reaches into the dialysis fluid circuit. The balance chamber 3 thereby balances between the fresh dialysis fluid and the used dialysis fluid, the dialysate. A mixed fluid sensor 13 is connected upstream of the dialyzer 9, in order to check the correct composition of the dialysis fluid. A bypass valve 17 is connected downstream from the mixed fluid sensor 13, for example formed as conductivity sensor.

The bypass valve 17 is opened, if the mixed fluid sensor 13 detects an unphysiological fluid, that is a fluid, which does not meet a predetermined condition, for example a predetermined conductivity, during the dialysis treatment.

To form the bypass, that is, the prevention of a fluid flow via a line branch 15 through the dialyzer 9, locking elements 91, 92 are operated. In particular a dialyzer intake valve 91 as well as a dialyzer drain valve 92, which control the intake and drain of the dialysis fluid to the dialyzer 9, are thereby closed. The dialysis fluid thus flows over a line portion 14. The valves can thereby be formed as magnetic valves. As a result, it is prevented that unphysiological fluid reaches the dialyzer 9. The safety of the patient is ensured.

In addition to the monitoring of the correct composition of the dialysis fluid, the operation of the blood treatment device can also be checked, for example for the presence of a leakage, so as to ensure the safety of the patient. To identify possible leakages in the system, a pressure holding test is performed. As part of this pressure holding test, deviations from a stable state can be captured by means of signal monitoring of at least one pressure sensor 16.

During the dialysis, the pressure holding test is performed at regular time intervals, for example every 12.5 minutes. For this purpose, the dialyzer 9 is disconnected from the dialysis fluid circuit for a certain time interval, for example 8 seconds. The line portion 14 is thus formed as a closed fluid system while performing the pressure holding test. To form this closed fluid system, the control unit controls the locking elements 91, 92 in such a way that they block the fluid flow. To form the closed fluid system, the balance chamber 3 is additionally held in a state, that is, no fluid flow takes place via the balance chamber, that is, no switch-over of the balance chamber 3.

When the dialyzer 9 is disconnected from the dialysis fluid circuit, the blood treatment device is thus in the bypass operation. As described above, the dialyzer intake valve 91 as well as dialyzer drain valve 92 are closed, while the bypass valve 17 is open. As a result, the line branch 15 is disconnected from the remaining fluid line system 10 by closing the corresponding valves. In other words, no fluid flow takes place between the line branch 15 and the remaining fluid line system 10 after the closing of the valves. To attain a test, which is as complete as possible, a change of the balance chamber half, which belongs to the line portion 14, takes place between two consecutive pressure holding tests.

In this case, this line portion 14 forms a self-contained system, in the case of which a stable pressure can be expected. Compared to the treatment pressure, the pressure prevailing in the line portion 14 during the pressure holding test is thereby increased. To detect possible leakages, the pressure curve during the pressure holding test is captured. If a pressure drop is detected, a conclusion can be drawn to the presence of a leakage.

Prior to the beginning of the pressure holding test, dialysis fluid is supplied from the supply system, and thus also via the concentration supply line 26, 36, until the desired pressure is reached. No fluid outflow takes place during the supply of the dialysis fluid into the line portion 14, so that pressure is built up. If the presence of a condition, for example a conductivity, which does not meet the expected value, is now measured by means of the mixed fluid sensor 13, a conclusion can be drawn to the presence of an unphysiological fluid in the dialysis fluid circuit.

If an unphysiological fluid is detected, the blood treatment device switches into the bypass operation, as described above, so as to prevent that unphysiological fluid reaches the dialyzer 9. After the detection of an unphysiological fluid, the dialysis fluid circuit can be flushed in a cleaning program. It can thus be prevented that unphysiological fluid, which is present in the dialysis fluid circuit, reaches into the dialyzer 9.

The formation of a closed system, for example for performing the pressure holding test, is prevented by means of the blood treatment device according to the invention, as soon as an unphysiological fluid is detected. The pressure holding test is thus prevented at the earliest possible point in time, or a pressure holding test, which has already been started, is terminated, respectively. As a result, the interruption of the treatment as a whole can be kept as short as possible.

As illustrated in FIG. 2, the blood treatment device has concentrate supply lines 26, 36 for supplying concentrate. The corresponding amount of concentrate is supplied via these concentrate supply lines 26, 36 to the permeate via the respective concentrate pump 25 or 36, respectively, and the desired composition of the dialysis fluid is thus attained.

The respective concentrate is supplied to the permeate as concentrate solution, that is, in liquid form. Concentrate sensors 23, 33 are in each case arranged in the respective concentrate supply lines 26, 36. The supply of the concentrate or the composition of the concentrate solution, respectively, can be monitored by means of these concentrate sensors 23, 33. The monitoring of the concentrate supply via concentrate supply lines 26, 36 can take place, for example, by means of conductance sensors or conductivity sensors.

The concentrate supply of the blood treatment device can take place from concentrate containers delivered to the blood treatment device, by means of connecting means 28, 38 inserted into said concentrate containers. In the arrangement illustrated in FIG. 2, the connecting means 28, 38 are inserted into chambers 27, 37 of the blood treatment device. This configuration, in which the connecting means 28, 38 are located in the chamber 27, 37, will be assumed for a flushing process of the blood treatment device. In addition, the position of the connecting means 28, 38 differs, depending on the concentrate supply mode, as will be described above. For the position capture, connecting means sensors 29, 39 are attached to the chambers 27, 37 of the blood treatment device in the embodiment shown in FIG. 2.

A corresponding concentrate solution in liquid form is prepared for the dialysis treatment in the concentrate containers, which are not illustrated in FIG. 2. This type of concentrate supply is understood here as first concentrate supply mode (KVM). In the alternative, the concentrate supply can be designed from bags, which are fastened to the blood treatment device, comprising dry concentrate or as central concentrate supply.

Variations can also be provided, in the case of which the concentrate supply for the respective concentrates takes place in different ways. For example, the concentrate supply of bicarbonate can be designed as dry concentrate, which is filled into bags, while the acid concentrate is delivered to the blood treatment device in liquid form in a concentrate container.

In the first case, in which the concentrate supply takes place from concentration solution provided in liquid form via concentrate containers, connecting means 28, 38, for example suction wands, are inserted into the respective concentrate containers. These connecting means 28, 38 supply the concentrate solution to the permeate via the concentrate supply line 26, 36.

For the second alternative of the concentrate supply by means of dry concentrate, the respective connecting means 28, 38 is inserted into a chamber 27, 37, for example a flushing chamber, of the blood treatment device. In the case of this alternative, a part of the fluid from the supply system, which flows in via the dialysis water connection 1, can be supplied to the bag comprising dry concentrate, controlled via a control valve.

After the fluid supply into the dry concentration bag, the concentrate solution, which is now fluid, is analogously supplied to the permeate via the chamber 27, 37, in which the respective connecting means 28, 38 is inserted. For this purpose, the bags comprising dry concentrate are fastened to the blood treatment device at corresponding interfaces, for example to protrusions formed with supply and discharge lines.

As further alternative, the concentrate supply of the respective concentrate can take place centrally. A concentrate container is thereby not provided at each blood treatment device. On the contrary, the supply takes place via a collection canister, which can supply several blood treatment device with concentrate. For this purpose, the individual blood treatment devices have a line connection to this collection canister. In this case of the concentrate supply, the respective connecting means 28, 38 is also inserted into the chamber 27, 37 of the blood treatment device.

In the case in which the concentrate supply takes place via dry concentrate in bags, it is detected that the concentrate solution is not supplied from the concentrate containers. This type of concentrate supply is detected, for example, by means of an additional sensor system. Contact sensors, for instance Hall sensors, can thereby be arranged on a housing cover of the blood treatment device, which is associated with the fastening of a dry concentrate bag. In the alternative, the type of the concentrate supply can be set manually on the blood treatment device. The connecting means 28, 38 remains inserted in the chamber 27, 37 during this type of concentrate supply.

In the case in which the concentrate supply takes place centrally, this can also be detected via a sensor system. For this purpose, a contact sensor can be provided at the corresponding connecting point for the connecting line of the blood treatment device to the central concentrate supply. In the alternative, the type of the concentrate supply can be selected manually on the blood treatment device.

Independently of the type of the concentrate supply, the concentrate flow takes place via the corresponding connecting means 28, 38 and the corresponding concentrate supply line 26, 36 to the permeate, wherein the concentrate flow passes through fluid sensors arranged in the concentrate supply lines 26, 36, more precisely the first concentrate sensor 23 and the second concentrate sensor 33. As described above, these concentrate sensors 23, 33, which are formed in the concentrate supply lines 26, 36, are sensors, which provide information relating to the concentrate solution prevailing in the concentrate supply lines 26, 36.

If the concentrate sensors 23, 33, which are arranged in the concentrate supply lines 26, 36, as illustrated in FIG. 2, are formed as conductance sensors, they detect whether a fluid is present in the concentrate supply line 26, 36. These conductance sensors can differentiate between a state—conductivity detected—and a state—no conductivity detected—and can thus provide insight into the presence of a concentrate flow. If the concentrate sensors 23, 33 emit a signal that no conductivity was detected, a start of the pressure holding test is prevented.

Due to the fact that the blood treatment device is already in bypass operation during the pressure holding test, a switch-over cannot take place any longer in the case of a capture of an unphysiological fluid, for example a detection of air in a concentrate supply line 26, 36 (state no conductivity detected). According to the claims, this signal is evaluated, however, in order to terminate a pressure holding test, which has already been started. It is prevented thereby that air reaches into the dialysis fluid circuit. If an unphysiological fluid can already be detected at this early point in time, the interruption of the dialysis treatment can be kept short. A pressure holding test is not performed when it is certain that the condition for a fluid value is not met. The condition for the fluid value is not met when an unphysiological fluid is present.

As described above, not only an already started pressure holding test is also terminated by evaluating the concentrate sensors 23, 33 as part of the pressure holding test, but the start of a pressure holding test is prevented as well. To keep interruptions of the dialysis treatment short, an evaluation of the concentrate sensors 23, 33 thus also takes place according to the claims as part of the pressure holding test.

As described above, pressure value sensors, which detect the type of the concentrate supply, can be provided In addition to the concentrate sensors 23, 33. By evaluating these sensors, it is possible to detect unphysiological fluid early and to effect a prevention of the pressure holding test. If, for example, the concentrate supply takes place via bags, which are filled with dry concentrate, this can be detected by means of contact sensors, for instance Hall sensors. A contact sensor can be arranged, for example, on a housing cover, which has to be operated in order to fasten the dry concentrate bag.

If the evaluation of the signal of a contact sensor shows that he housing cover is open, a conclusion can be drawn to a concentrate supply with dry concentrate. To detect the central concentrate supply, a contact sensor can similarity be formed at an interface for connecting the central hose system. In the alternative or in addition, the type of the concentrate supply can be selected manually at the blood treatment device.

If a concentrate supply via concentrate containers is present, the respective connecting means 28, 38 can be inserted into the corresponding concentrate container for this purpose. If it is captured, however, that the respective connecting means 28, 38 is located in the chamber 27, 37, it results from this fact that no physiological fluid can be drawn in. In the case of this further termination condition, it can be detected first, which type of the concentrate supply is present.

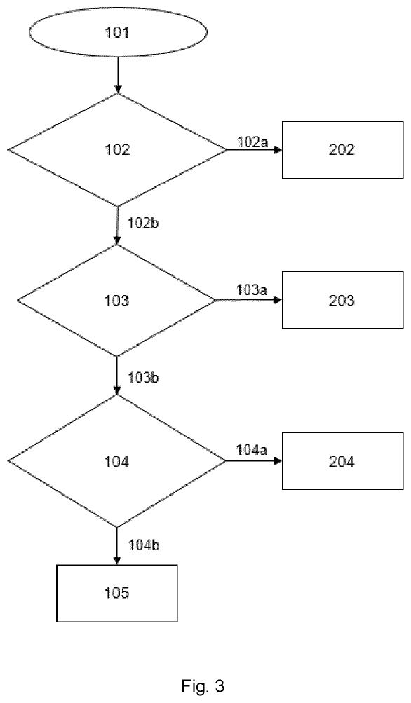

The position of the corresponding connecting means 28, 38 is determined subsequently, in order to capture whether the connecting means is inserted into the concentrate container or into the chamber 27, 37 of the blood treatment device or is located therein, respectively. This can take place, for example, via an evaluating of contact sensors. An exemplary course of this test sequence is illustrated in FIG. 3.

Step 101 thereby indicates the beginning of the pressure holding test. The query whether the fluid value meets the predetermined condition takes place in step 102. If the fluid value meets the predetermined condition (102b), it is determined in step 103, whether the concentrate supply mode is a first concentrate supply mode. If this condition is met as well (103b), it is determined in step 104, whether the position of the connecting means 28, 38 meets the predetermined condition. If the determination of the corresponding condition provides a positive result (102b, 103b, 104b), the pressure holding test is performed in step 105.

If the respective condition is not met, that is, if the fluid value does not meet the predetermined condition, the pressure holding test is terminated in step 202. If the condition is not met that the concentrate supply mode is a first concentrate supply mode, the pressure holding test is likewise terminated in step 203. If the condition is not met that the position of the connecting means 28, 38 meets a predetermined condition, the pressure holding test is likewise terminated in step 204.

The order of the steps can be changed thereby, so that the position of the connecting means 28, 38 is detected first, and subsequently the type of the concentrate supply.

In addition to the conditions illustrated in FIG. 3, further conditions can be captured in order to make a decision to perform the pressure holding test. Not all conditions illustrated in FIG. 3 have to likewise be queried. It may be sufficient, for example, to check only a presence of the condition for the fluid value.

If it is thus detected that the corresponding connecting means 28, 38 is inserted into the chamber 27, 37 of the blood treatment device and not into the concentrate container, a conclusion can be drawn therefrom that the connecting means 28, 38 draws in unphysiological fluid. To also prevent that unphysiological fluid is drawn in and reaches into the dialysis fluid circuit in this case, the pressure holding test is prevented or terminated, respectively.

If it is detected, in contrast, that a central concentrate supply or a dry concentrate supply is present, in other words a concentrate supply, in the case of which the connecting means 28, 38 has to be inserted into the chamber 27, 37 of the blood treatment device, a termination of the pressure holding test does precisely not take place.

To detect whether the connecting means 28, 38 is inserted into the chamber 27, 37 of the blood treatment device, connecting means sensor 29, 39 or position detecting sensors, respectively, can in each case be assigned to the respective chambers 27, 37, into which the connecting means 28, 38 are inserted, as described with regard to FIG. 2. For example magnetic sensors or contact sensors, for instance Hall sensors, can be used for this purpose. In the alternative, mechanical switches, for instance pressure switches, toggle or rocker switches can be used.

By evaluating this additional information, the type of the concentrate supply, for example obtained by means of connecting means sensors 29, 39 as well as fluid sensors 13, 23, 33, in particular concentrate sensors in the concentrate supply lines 26, 36 as part of the pressure holding test, unphysiological fluids can be detected at an early point in time, more exactly prior to reaching the dialysis fluid circuit. The pressure holding test is thus not performed in the case that the predetermined conditions are not met. The predetermined conditions are not met when an unphysiological fluid is present.

As a result, interruptions of the dialysis treatment are kept short in that no closed system is formed when the necessary conditions are not met. In the case of a corresponding control, a time-intensive flushing, for example, can additionally be prevented, in order to clean the dialysis fluid circuit. In addition, the patient safety is increased by preventing the pressure holding test when an unphysiological fluid is present.

If the pressure holding test is performed, higher pressures as compared to the pressures, which are present during a treatment, are produced in the dialysis fluid circuit. If unphysiological fluid is present in the dialysis fluid circuit, it can be prevented in any case by means of the solution according to the claims that the pressure holding test is performed and high pressures are generated.

The control can have a processor or a microchip, respectively, which, in combination with a storage device, in which a program code is stored, can be configured or programmed, respectively, to perform the corresponding controls in the blood treatment device. The processor or the microchip, respectively, is set up to process data and/or to take over the communication, inter alia. The processor or the microchip, respectively, can be programmed by means of configuration presets, and then, inter alia, also serves as processing unit for processing operational and machine data.

1. A blood treatment device, having
a fluid line system for guiding a fluid flow comprising a line portion, wherein the line portion is capable of being a closed fluid system, and comprising at least one first concentrate supply line for supplying a first concentrate solution;
a fluid pump for conveying a fluid in the fluid line system;
a determining means for capturing a state of the fluid line system;
a control unit for controlling the fluid flow;
wherein the control unit is configured in such a way that the line portion only forms a closed fluid system when the state captured by the determining means meets a predetermined condition.

2. The blood treatment device according to claim 1, further having
a first concentrate sensor for capturing a first concentrate value prevailing in the first concentrate supply line, wherein the determining means comprises the first concentrate sensor, and the predetermined condition is only met when the first concentrate value meets a first concentrate value condition; and/or
a mixed fluid sensor for capturing a mixed fluid value prevailing in the fluid line system downstream from the first concentrate supply line, wherein the determining means comprises the mixed fluid sensor, and the predetermined condition is only met when the mixed fluid value meets a mixed fluid value condition.

3. The blood treatment device according to claim 1, further having
a connecting means for transferring the first concentrate solution into the first concentrate supply line;
a chamber, into which the connecting means is capable of being introduced;
a connecting means sensor for capturing a position of the connecting means; and wherein
the determining means comprises the connecting means sensor, and the predetermined condition is only met when the position of the connecting means meets a position condition.

4. The blood treatment device according to claim 3, further having
a means for capturing a concentrate supply mode, wherein
the predetermined condition is only met when a position condition is met, and when the captured concentrate supply mode is a concentrate supply mode, in which the concentrate supply takes place via the connecting means in the detected position.

5. The blood treatment device according to claim 1, further having
a second concentrate supply line for supplying a second concentrate solution, and
a second concentrate sensor for capturing a second concentrate value in the second concentrate supply line, wherein the determining means comprises the second concentrate sensor, and
the predetermined condition is only met when the second concentrate value meets a second concentrate value condition.

6. The blood treatment device according to claim 3, wherein
the connecting means sensor has a magnetic sensor or a contact sensor.

7. The blood treatment device according to claim 3, wherein
the connecting means sensor is arranged on the chamber of the blood treatment device and/or the connecting means sensor is formed in such a way to be capable of being attached to a concentrate container, which can be delivered to the blood treatment device.

8. The blood treatment device according to claim 1, wherein
the first and/or second concentrate sensor is a conductance sensor or a conductivity sensor or an ultrasonic sensor, and/or the mixed fluid sensor is a conductance sensor or a conductivity sensor or an ultrasonic sensor.

9. The blood treatment device according to claim 1, further having
a line branch, which is formed parallel to at least one partial portion of the line portion, so that a fluid flow can be connected via the line branch in the case of a fluid flow via the line portion; and
a dialyzer in the line branch.

10. The blood treatment device according to claim 1, further having
locking elements in the fluid line system, wherein
the control unit is configured so as to form the line portion as a closed fluid system by controlling at least the locking elements.

11. The blood treatment device according to claim 1, further having
a pressure sensor for capturing a pressure value in the line portion, wherein
the control unit is thereby configured to only perform a pressure holding test in the line portion when the state captured by the determining means meets the predetermined condition,
in which the pressure holding test involves applying a pressure to the fluid in the line portion, and the pressure values in the line portion are captured during a predetermine time period, and a conclusion is drawn from a change of the pressure values to a leakage in the line portion.

12. The blood treatment device according to claim 1, further having
at least one first concentrate pump for conveying the first concentrate solution.

13. A method for controlling a fluid flow in a blood treatment device, wherein the blood treatment device has a fluid line system for guiding a fluid flow comprising a line portion, which can be formed as a closed fluid system, and at least a first concentrate supply line for supplying a first concentrate solution; having
detecting a state of the fluid line system by means of a determining means; and
comparing the state to a predetermined condition;
wherein
allowing that the line portion only forms a closed fluid system when the state of the fluid line system meets the predetermined condition.

14. The method according to claim 13, wherein the blood treatment device has a first concentrate sensor as determining means for capturing a first concentrate value prevailing in the first concentrate supply line, and wherein the predetermined condition is only met when the concentrate value meets a concentrate value condition and/or the blood treatment device has a mixed fluid sensor as determining means for capturing a mixed fluid value in the fluid line system downstream from the concentrate supply line, and wherein the predetermined condition is only met when the mixed fluid value meets a mixed fluid value condition.

15. The method according to claim 13, wherein the blood treatment device has a chamber and a connecting means, which can be introduced into the chamber, as well as a connecting means sensor as determining means for capturing a position of the connecting means, further having
detecting a position of the connecting means by means of the connecting means sensor, wherein
the predetermined condition is only met when the position of the connecting means meets a position condition.

16. The blood treatment device according to claim 3, wherein the connecting means sensor is a Hall sensor or a mechanical switch.

17. The blood treatment device according to claim 11, wherein the pressure is 600 to 800 mmHg.

18. The blood treatment device according to claim 11, wherein the pressure is from 700 to 750 mmHg.
